(19) United States
(12) Patent Application Publication
(10) Pub. No.: US 2024/0325825 A1
(43) Pub. Date: Oct. 3, 2024

(54) PROSTHETIC FIN

(71) Applicant: FINIS INC., Livermore, CA (US)

(72) Inventor: David F. Beittel, San Leandro, CA (US)

(73) Assignee: FINIS INC., Livermore, CA (US)

(21) Appl. No.: 18/445,075

(22) Filed: Mar. 29, 2023

(51) Int. Cl. A63B 31/12

(52) U.S. Cl. CPC A63B 31/12; A61F 2002/6614

(57) ABSTRACT

A fin that includes a flared fin body and a strap feature that is configured to wrap around a residual limb or an amputee thereby allowing the amputee to fin kick in an aquatic environment with the fin attached to the residual limb. The strap feature is preferably perpendicular to the elongated direction of the flared fin body and can include multiple band structures along with any number or release mechanisms. The back portion of the fin is open and void of obstruction allowing the residual limb of the amputee to be inserted under the strap feature and with the flared fin body cupping the residual limb.

### FIELD OF THE INVENTION

The present invention relates to fins used in water related activities including swimming training, snorkeling and scuba diving.

### BACKGROUND ON INVENTION

Swim fins and dive fins are worn on feet. Fins are usually made from rubber, plastic or combinations of these materials, to aid movement through the water in water sports activities such as swimming, body-boarding, body-surfing, knee-boarding, river-boarding, underwater hockey, underwater rugby, snorkeling and scuba diving.

Fins help the wearer to move through water more efficiently, as human feet are too small and inappropriately shaped to provide much thrust, especially when the wearer is carrying equipment that increases hydrodynamic drag. Very long fins and mono-fins provide underwater propulsion and do not require high frequency leg movement. This improves efficiency and helps to minimize oxygen consumption. Short, stiff-bladed fins are effective for short bursts of acceleration and maneuvering.

All swim fins and dive fins that are currently available are designed to secure to a users fee. For example, some swim fins and dive fins have full-boot foot packets, wherein feet are inserted into the full-boot foot packets and the full-boot foot packets completely surround the feet to thereby secure the swim fins or dive fins in place. Other fins have partial-boot foot packets and ankle straps, wherein feet are inserted into the partial-boot foot packets and the ankle straps are tightened behind ankles to thereby secure the swim fins or dive fins in place.

### SUMMARY OF THE INVENTION

A fin for swimming or diving that attaches to a residual limb of an amputee is disclosed. The fin of the present invention can be configured to attach to a residual arm limb of an amputee or a residual leg limb of the amputee.

The fin includes a fin body. The fin body is preferably contoured or curved in the longitudinal direction to cup the residual limb and flares or broadens towards the front end of the fin body. The front portion of the flared fin body can includes a center contoured membrane that is configured to deform when the amputee kicks or strokes the fin in an aquatic environment with the fin attached to the residual limb of the amputee.

Attached to the flared fin body is a perpendicular strap feature that is configured to wrap around the residual limb and secure the flared fin body thereto. The strap features can be a dual band structure, a single band or an tube with or without straps. Where a strap or straps are used, the strap features preferably have a two-part clip features for quickly releasing the fin from the residual limb of the amputee.

### BRIEF DESCRIPTION OF THE DRAWINGS

FIGS. 1A-B show perspective views of the fin for attaching to a residual limb of an amputee, in accordance with the embodiments of the invention.

FIG. 2A shows a bottom perspective view of the fin for attaching to a residual limb of an amputee, in accordance with the embodiments of the invention.

FIG. 2B shows a top view of the fin for attaching to a residual limb of an amputee, in accordance with the embodiments of the invention.

FIG. 2C shows a side view of the fin for attaching to a residual limb of an amputee, in accordance with the embodiments of the invention.

FIG. 2D shows a bottom view of the fin for attaching to a residual limb of an amputee, in accordance with the embodiments of the invention.

FIG. 2E shows a front view of the fin for attaching to a residual limb of an amputee, in accordance with the embodiments of the invention.

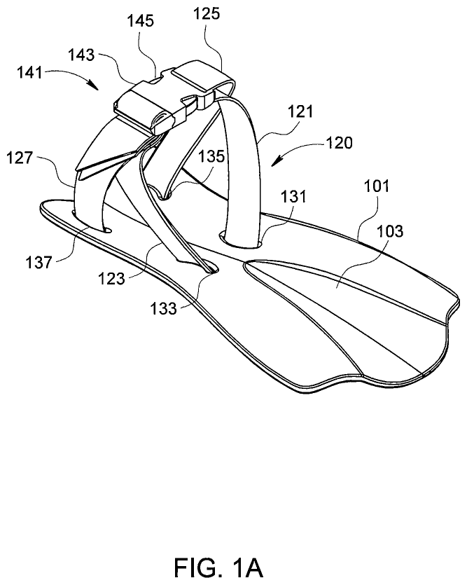

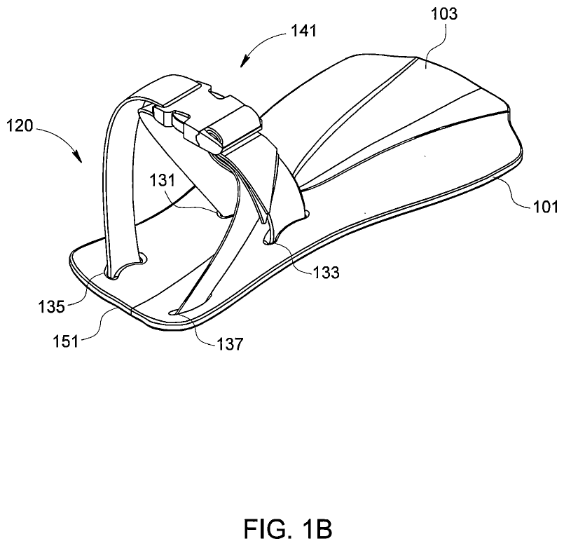

### DETAILED DESCRIPTION OF THE INVENTION

The invention is directed to a fin 120 for swimming or diving that attaches to a residual limb of an amputee (not shown), The fin 120 of the present invention can be configured to attach to a residual arm limb of an amputee or a residual leg limb of the amputee. The fin 120 includes a fin body 101 and a perpendicular strap feature 141. The fin body 101 is preferably contoured or curved in the longitudinal direction to cup the residual limb and flares or broadens towards the front end of the fin body 101. The front portion of the flared fin body 101 can includes a center contoured membrane 103 that is configured to deform when the amputee kicks or strokes the fin 120 in an aquatic environment with the fin 120 attached to the residual limb. The back portion of the fin 120 is open and void of obstruction allowing the residual limb of the amputee to be inserted under the strap feature 141 and with the fin body 101 cupping the residual limb.

The flared fin body 101 is preferably molded from a polymeric material such a rubber or plastic including, but not limited to, polyurethane or polypropylene. The center contoured membrane 103 can be co-molded with remaining portion of flared fin body 101 and can be formed from the same material or a different material. The center contoured membrane 103 can also be thinner than the remaining portion of flared fin body 101 allowing the center contoured membrane 103 to flex up and down when the amputee kicks or strokes the fin 120 in the aquatic environment within the fin 120 attached to the residual limb.

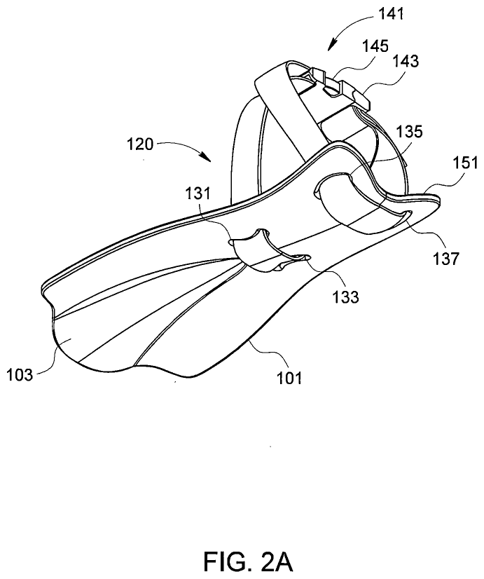

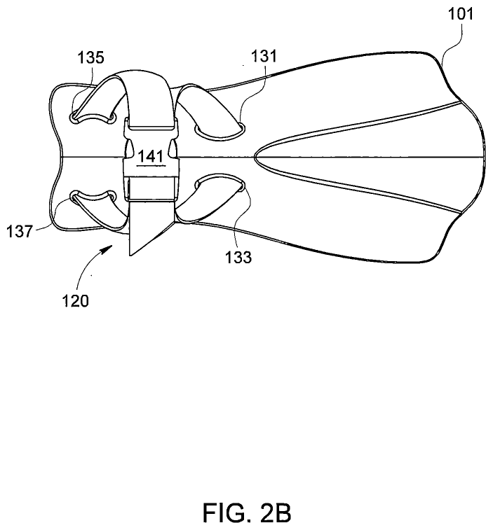

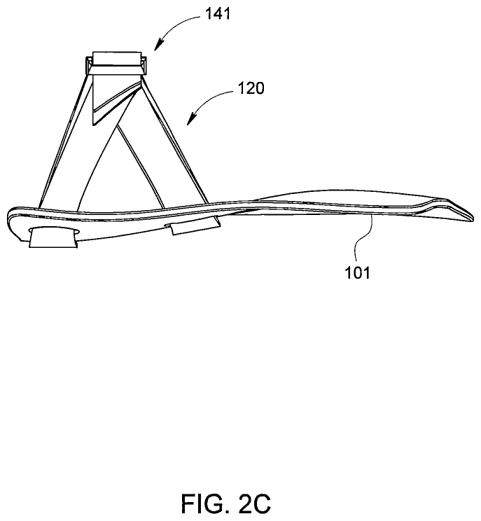

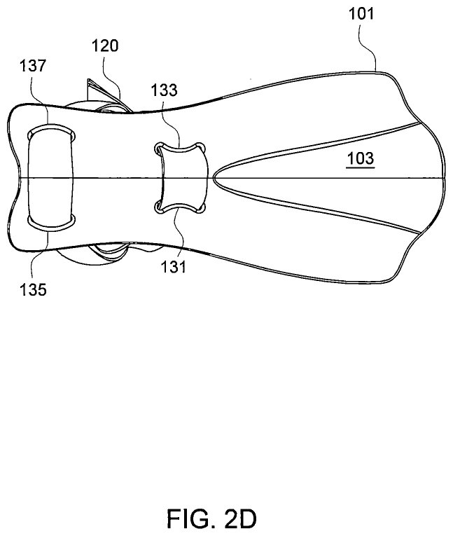

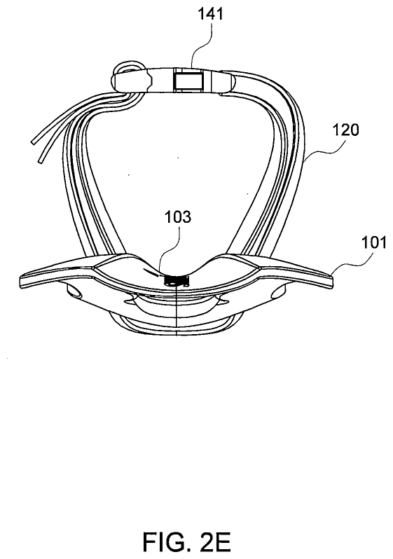

Attached to the flared fin body 101 is a perpendicular strap feature 141 that is configured to wrap around the residual limb and secure the flared fin body 101 thereto. The perpendicular strap feature 141 can be a dual band structure with two or more band structures 121 and 127 being fitted through slotted apertures 131/133 and 135/137 and wrapping around bottom portions 151 of the flared fin body 101, as shown in FIG. 2A.

The dual band structure 141 preferably include a two-part clip features 143/145 with a quick release mechanism that connects a right side 123 of the dual band structure 141 to a left side 125 of the dual band structure 141 and secures flared fin body 101 to the residual limb, as described above.

The dual band structure 141 can be made from any suitable material or materials including rubber, plastic, silicone or any other suitable polymeric material. The dual band structure 141 can also be formed from a woven material or fabric. Regardless of the material used to form the dual band structure 141, the dual band structure can be a monolithic strap structure that loops twice underneath the flared fin body 101 (as shown in FIG. 2A) through four slotted apertures 131/133 and 135/137 and connect to single attachment positions on each portion of the two-part clip features 143 and 145. The portions of the two-part clip features 143 and 145 are formed form any suitable material including plastic, metal, such as aluminum or any other suitable material or materials.

The fin body 101 can be a split fin body and the perpendicular strap feature can be a single strap structure having any suitable width to secured around the residual limb of the amputee using any suitable meanings including, but not limited to, velcro, buckles, two-part snap features and combinations thereof. In accordance with further embodiments of the invention, the band structure is and elastic tube sock structure with traction surfaces that pressure fits around the residual limb of the amputee and secures thereto through friction/pressure. The elastic tube sock structure can be attached to the fin body by any suitable means and can be used alone or in combination with strap structures, such as the strap structures described above.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. A fin for attaching to a residual limb of an amputee comprising:
a) a flared fin body; and
b) a perpendicular strap feature that is configured to wrap around the residual limb and secure the flared fin body thereto.

2. The fin of claim 1, wherein the perpendicular strap feature has a dual band structure.

3. The fin of claim 2, wherein the dual band structure further comprising a two-part clip features with a quick release mechanism that connects a right side of the dual band structure to a left side of the dual band structure.

4. The fin of claim 3, wherein the dual band structure attached to the flared fin body through slotted apertures and wherein portions of the dual band structure fit through the slotted apertures and wrap around bottom portions the flared fin body.

5. The fin of claim 1, wherein the flared fin body contoured or curved in the longitudinal direction to cup the residual limb and flares or broadens towards the front end of the flared fin body.

6. The fin of claim 5, further comprising a center contoured membrane that is configured to deform when the amputee kicks or strokes the fin in an aquatic environment with the fin attached to the residual limb.

7. A fin for attaching to a residual limb of an amputee comprising:
a) a flared fin body that is contoured or curved in the longitudinal direction to cup the residual limb and flares or broadens towards the front end of the flared fin body; and
b) a perpendicular strap feature attached to the flared fin body that is configured to wrap around the residual limb and secure the flared fin body thereto.

8. The fin of claim 7, further comprising a center contoured membrane that is configured to deform when the amputee kicks or strokes the fin in an aquatic environment with the fin attached to the residual limb.

9. The fin of claim 7, wherein the perpendicular strap feature is a dual band structure.

10. The fin of claim 7, the band structure further comprising a two-part clip features with a quick release mechanism that connects a right side of the dual band structure to a left side of the dual band structure.

11. The fin of claim 7, wherein the strap feature is attached to the flared fin body through slotted apertures, wherein portions of the strap feature fit through the slotted apertures and wrap around bottom portions the flared fin body.

12. The fin of claim 7, further comprising a center contoured membrane that is configured to deform when the amputee kicks or strokes the fin in an aquatic environment with the fin attached to the residual limb.

13. A fin for attaching to a residual limb of an amputee comprising:
a) a flared fin body flared fin body contoured or curved in the longitudinal direction to cup the residual limb and flares or broadens towards the front end of the flared fin body and having a center contoured membrane that is configured to deform when the amputee kicks or strokes the fin in an aquatic environment with the fin attached to the residual limb; and
b) a perpendicular strap feature attached to the flared fin body through slotted apertures, the strap feature being a dual band structure with a two-part clip features that connects a right side of the dual band structure to a left side of the dual band structure and is configured to wrap around the residual limb and secure the flared fin body thereto.
